(19) United States
(12) Patent Application Publication
(10) Pub. No.: US 2021/0338202 A1
(43) Pub. Date: Nov. 4, 2021

(54) SYSTEM AND METHOD FOR CONTROLLING AN ACTIVATION STATE OF AN ULTRASOUND PROBE

(71) Applicant: GE Precision Healthcare LLC, Wauwatosa, WI (US)

(72) Inventors: Gerhard Hattinger, Pramet (AT); Markus Paarhammer, Oberwang (AT)

(21) Appl. No.: 16/862,206

(22) Filed: Apr. 29, 2020

(51) Int. Cl. A61B 8/00; A61B 8/08

(52) U.S. Cl. CPC A61B 8/4483; A61B 8/4477; A61B 8/5207

(57) ABSTRACT

Systems and methods are provided for controlling an activation state of an ultrasound probe that is part of an ultrasound imaging system. The ultrasound probe includes a cable, and the ultrasound imaging system includes a probe holder defining a slot for allowing passage of a cable. The activation state of the ultrasound probe is controlled by a processor based on signals received from a sensor assembly attached to the probe holder and configured to detect movement of the cable through the slot defined by the probe holder.

### FIELD OF THE INVENTION

This disclosure relates generally to an ultrasound imaging system and method for controlling an activation state of an ultrasound probe.

### BACKGROUND OF THE INVENTION

Ultrasound imaging systems having more than one ultrasound probe must provide a technique for activating and deactivating a particular ultrasound probe. A first existing technique for activating and deactivating a particular ultrasound probe includes manually activating and deactivating the desired ultrasound probe by means of pushing a particular button on the ultrasound imaging system that corresponds to the desired ultrasound probe. A second existing technique includes using a switch located on the ultrasound probe itself and manually pressing the switch to activate or deactivate the ultrasound probe. A third existing technique includes placing a mechanical switch in a location on the probe holder that contacts the ultrasound probe when the ultrasound probe is placed in the probe holder. The mechanical switch is deactivated when the ultrasound probe is in contact with the mechanical switch. The mechanical switch is activated when the ultrasound probe is not in contact with the mechanical switch. The ultrasound imaging system activates the corresponding ultrasound probe when the switch is activated and deactivates the corresponding ultrasound probe when mechanical switch is deactivated. A fourth existing technique for activating and deactivating an ultrasound probe includes fitting motion sensors on the ultrasound probes. The ultrasound system activates or deactivates the ultrasound probe in response to the motion signals received from the motion sensors.

There are disadvantages to each of the techniques described above. The first technique and second technique each have the disadvantage of requiring the user to push a button to indicate the particular ultrasound probe to be activated or deactivated. Additionally, the user may need to trace the ultrasound probe back to the corresponding probe port in order to activate the correct ultrasound probe. The third technique has the disadvantage of false activations or deactivations of the ultrasound probe. Users often place gel bottles, cell phones, or other objects in the probe holders. Objects other than ultrasound probes can activate or deactivate the switch on the probe holder, leading to undesired activations or deactivations of the ultrasound probe. The third technique also requires the ultrasound probe to be returned to the probe holder before another ultrasound probe can be activated by the system. The third technique also requires the ultrasound probe to be returned to the probe holder from which it was removed in order to ensure the correct ultrasound probe is activated when removed from the probe holder. The fourth technique has the disadvantage of having to modify every ultrasound probe used by the ultrasound imaging system. A motion sensor must be attached to every ultrasound probe connected to the ultrasound imaging system, and to the ultrasound imaging system itself. An ultrasound probe will not automatically activate or deactivate until the new ultrasound probe has a motion sensor and the new motion sensor has been added to the system. This adds cost and complexity to each ultrasound probe. Additional resources are also necessary to track the motion of the ultrasound system, the ultrasound probes, and the movement of the ultrasound system in relation to the ultrasound probes. For at least the reasons discussed hereinabove, there is a need for an improved method and ultrasound imaging system for controlling an activation state of an ultrasound probe.

### BRIEF DESCRIPTION OF THE INVENTION

The above-mentioned shortcomings, disadvantages, and problems are addressed herein which will be understood by reading and understanding the following specification.

In accordance with an embodiment, an ultrasound imaging system includes an ultrasound probe including a cable and a probe holder including a cradle for supporting the ultrasound probe. The probe holder defines a slot for allowing passage of the cable and includes a sensor assembly attached to the probe holder. The sensor assembly is configured to detect movement of the cable through the slot and the sensor assembly includes at least one sensor. A processor is configured to control an activation state of the ultrasound probe based on signals from the sensor assembly.

In accordance with an embodiment, an ultrasound imaging system includes a processor, probe holder including a cradle for supporting the ultrasound probe and defining a slot for allowing passage of the cable. The ultrasound imaging system includes a sensor assembly attached to the probe holder that is configured to detect movement of the cable through the slot. A method for controlling an activation state of an ultrasound probe includes receiving a signal from the sensor assembly in response to detecting movement of the cable through the slot due to either removing the ultrasound probe from the cradle or placing the ultrasound probe in the cradle and changing the activation state of the ultrasound probe based on the signal.

Various other features, objects, and advantages of the invention will be made apparent to those skilled in the art from the accompanying drawings and detailed description thereof.

### BRIEF DESCRIPTION OF THE DRAWINGS

FIG. 1 is an illustration of an ultrasound imaging system in accordance with an embodiment;

FIG. 2 is schematic representation of an ultrasound imaging system in accordance with an embodiment;

FIG. 3 is an illustration of a probe holder in accordance with an embodiment;

FIG. 4 is an illustration of a sectional view of a probe holder in accordance with an embodiment;

FIG. 5 is an illustration of a probe holder in accordance with an embodiment;

FIG. 6 is an illustration of a probe holder in accordance with an embodiment;

FIG. 7 is an illustration of a sectional view of a probe holder in accordance with an embodiment;

FIG. 8 is an illustration of a probe holder in accordance with an embodiment;

and

FIG. 9 is an illustration of a sectional view of a probe holder in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized, and logical, mechanical, electrical and other changes may be made without departing from the scope of the embodiments. The following detailed description is, therefore, not to be taken as limiting the scope of the invention.

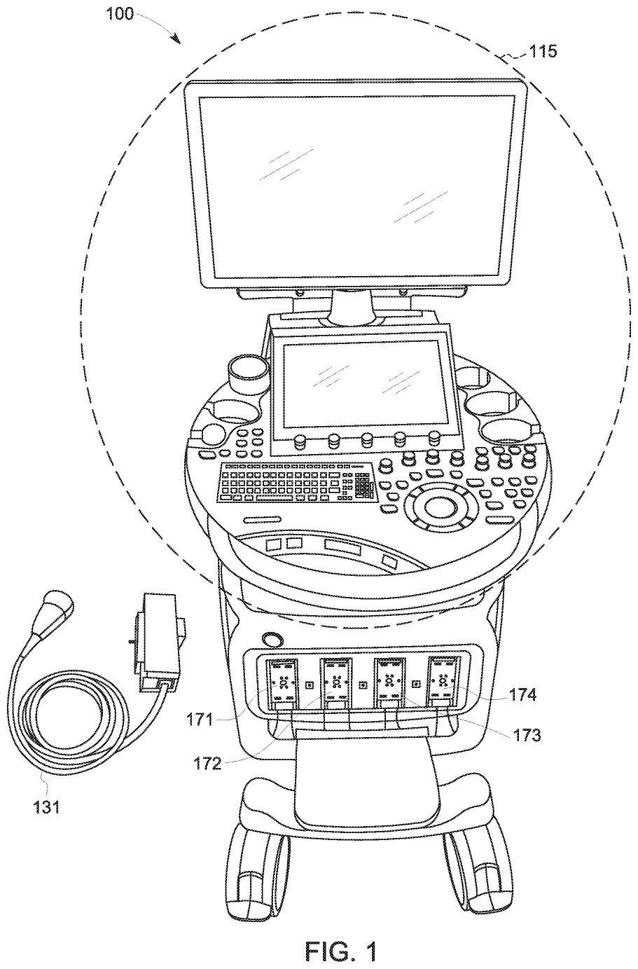

FIG. 1 illustrates an exemplary embodiment of an ultrasound imaging system 100. The ultrasound imaging system 100 includes a user interface 115, a first ultrasound probe 131 and a plurality of probe ports: a first probe port 171, a second probe port 172, a third probe port 173, and a fourth probe port 174. Each of the plurality of probe ports (171, 172, 173, 174) is configured to receive an ultrasound probe, such as the first ultrasound probe 131. The first ultrasound probe 131 may be connected to the ultrasound imaging system 100 by plugging the ultrasound probe 131 into one of the plurality of probe ports (171, 172, 173, 174).

The user interface 115 may be used to control operation of the ultrasound imaging system 100. For example, the user interface 115 may be used to control the input of patient data and/or to change a scanning or display parameter, and the like. The user interface 115 may include a plurality of user inputs and/or controls that are configured to receive commands from a user or operator. For example, the ultrasound imaging system 100 may include any type of user input control including one or more of: a mouse, a trackball, a keyboard, a touch pad, a touchscreen-based user interface, one or more hard buttons, sliders, rotaries, or any other type of physical control. The ultrasound imaging system 100 may further include one or more display devices. The display devices may be a touch screen display, a LED display, an OLED display, a liquid crystal display (LCD) a projection display device, or any other type display configured for displaying one or more images and/or capable of accepting user input. The display device may be a touch screen display, providing soft button implementations of the plurality of the various user inputs and controls. The user interface 115 further includes one or more probe holders for supporting the ultrasound probes. The number of probe holders provided is typically equal to or greater than the number of probe ports, but the number of probe holders may also be a smaller number than the number of probe ports.

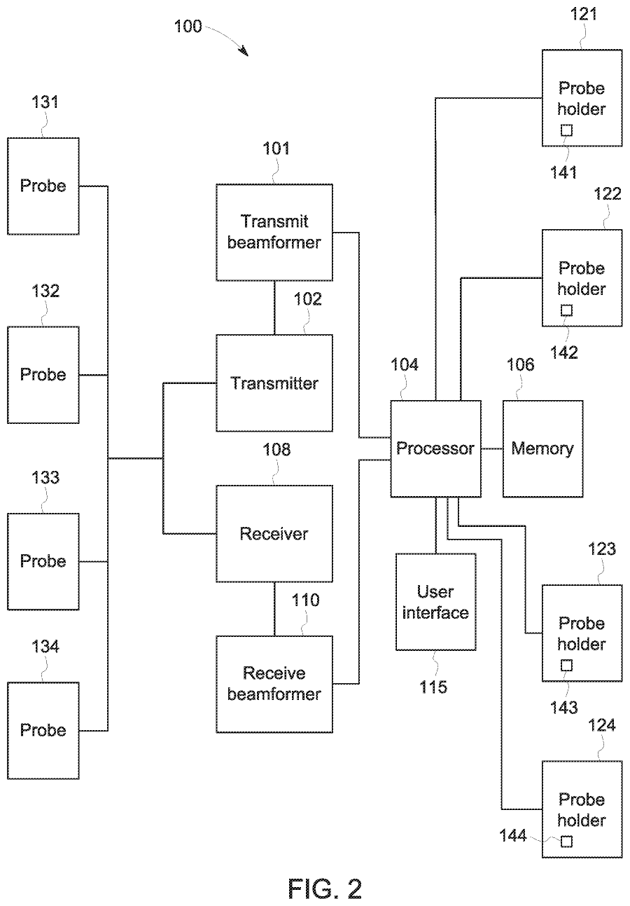

FIG. 2 is a schematic diagram of an ultrasound imaging system 100 in accordance with an embodiment. The ultrasound imaging system 100 includes a transmit beamformer 101 and a transmitter 102 configured to drive transducer elements (not shown) within a probe, such as the first probe 131, to emit pulsed ultrasonic signals into a body (not shown). The transducer elements are configured to both transmit and receive ultrasound signals. The pulsed ultrasonic signals are back-scattered from structures in the body, like blood cells or muscular tissue, to produce echoes that return to the transducer elements. The echoes are converted into electrical signals, or ultrasound data, by the transducer elements and the electrical signals are received by a receiver 108. The electrical signals representing the received echoes are passed through a receive beamformer 110 that outputs ultrasound data. According to some embodiments, an ultrasound probe, such as the ultrasound probe 131, may contain electronic circuitry to do all or part of the transmit and/or the receive beamforming. For example, all or part of the beamformer 101, the transmitter 102, the receiver 108 and the receive beamformer 110 may be situated within an ultrasound probe, such as the first ultrasound probe 131, according to an embodiment. The terms “scan” or “scanning” may also be used in this disclosure to refer to acquiring data through the process of transmitting and receiving ultrasonic signals. The terms “data” or “ultrasound data” may be used in this disclosure to refer to either one or more datasets acquired with an ultrasound imaging system. A user interface 115 may be used to control operation of the ultrasound imaging system 100, including the input of patient data and/or the selection of scanning or display parameters.

The ultrasound imaging system 100 further includes a processor 104 to control the transmit beamformer 101, the transmitter 102, the receiver 108, the receive beamformer 110, a memory 106, a second ultrasound probe 132, a third ultrasound probe 133, and a fourth ultrasound probe 134. The ultrasound imaging system 100 may also include: a first probe holder 121 including a sensor assembly 141; a second probe holder 122 including a sensor assembly 142; a third probe holder 123 including a sensor assembly 143; and a fourth probe holder 124 including a sensor assembly 144.

The processor 104 is in electronic communication with the transmit beamformer 101, the transmitter 102, the receiver 108, and the receive beamformer 110. The processor 104 is also in electronic communication with the plurality of ultrasound probes (131, 132, 133, 134), the plurality of sensor assemblies (141, 142, 143, 144), the memory 106, and the user interface 115. The processor 104 may control the plurality of ultrasound probes (131, 132, 133, 134) to acquire data. The processor 104 controls which of the transducer elements are active and the shape of a beam emitted from one of the plurality of ultrasound probes (131, 132, 133, 134). The processor 104 is also in electronic communication with the user interface 115, and the processor 104 may process the data into images for display on the user interface 115. For purposes of this disclosure, the term “electronic communication” may be defined to include both wired and wireless connections. The processor 104 may include a central processing unit (CPU) according to an embodiment. According to other embodiments, the processor 104 may include other electronic components capable of carrying out processing functions, such as a digital signal processor (DSP), a field-programmable gate array (FPGA) or a graphics board. According to other embodiments, the processor 104 may include multiple electronic components capable of carrying out processing functions. For example, the processor 104 may include two or more electronic components selected from a list of electronic components including: a central processing unit (CPU), a digital signal processor (DSP), a field-programmable gate array (FPGA), and a graphic board. According to another embodiment, the processor 104 may also include a complex demodulator (not shown) that demodulates the RF data and generates raw data. In another embodiment the demodulation may be carried out earlier in the processing chain. The processor 104 may be adapted to perform one or more processing operations on the data according to a plurality of selectable ultrasound modalities. The data may be processed in real-time during a scanning session as the echo signals are received. For the purposes of this disclosure, the term “real-time” is defined to include a procedure that is performed without any intentional delay. The memory 106 may include volatile or non-volatile memory of one or more of the following elements: a random-access memory (RAM), a read only memory (ROM), programmable read only memory (PROM), dynamic RAM (DRAM), static RAM (SRAM), rewritable flash, rewritable, byte addressable, symmetric, or any other type of electronic storage of information.

Each of the ultrasound probes (131, 132, 133, 134) may be the same type of ultrasound probe, or the ultrasound probes may include two or more different types of ultrasound probes. For example the ultrasound probes may include two or more types of ultrasound probes selected from the following group: a linear ultrasound probe, a convex ultrasound probe, a phased array ultrasound probe, or any other type of ultrasound probe capable of transmitting and receiving ultrasound sound pulses. Each of the plurality of ultrasound probes (131, 132, 133, 134) may be connected to any one of the plurality of probe ports (171, 172, 173, 174). Each of the plurality of probe holders (121, 122, 123, 124) may be specialized to hold a specific type of ultrasound probe or each of the plurality of probe holders (121, 122, 123, 124) may be configured to hold different types of ultrasound probes.

The processor 104 is in electronic communication with the plurality of probe ports (171, 172, 173, 174), the plurality of ultrasound probes (131, 132, 133, 134) that are connected to the plurality of probe ports (171, 172, 173, 174), the memory 106, the user interface 115, and the plurality of sensor assemblies (141, 142, 143, 144). The processor 104 receives signals from one of the sensor assemblies (141, 142, 143, 144) indicating the detection of a cable, such as a cable of the ultrasound probe 131, passing through a slot (not shown in FIG. 1 or FIG. 2) of one of the plurality of probe holders (121, 122, 123, 124) as the ultrasound probe 131 is removed from or placed in one of the probe holders. The processor 104 may also store, in the memory 106, the configuration information for each of the plurality of probe ports (171, 172, 173, 174), including the current state of each probe port as either having or not having an ultrasound probe connected. When an ultrasound probe is connected, the processor 104 may also store the activation state of the ultrasound probe, the ultrasound probe type, and the ultrasound probe identification (I.D.), and the probe holder to which the ultrasound probe is connected. The processor 104 also stores, in the memory 106, the configuration of the plurality of sensor assemblies (141, 142, 143, 144) for each probe holder. Each sensor assembly includes one or more sensors capable of detecting the passing of the cable through the slot of the probe holder. When a sensor assembly includes a single sensor, the sensor assembly may not be configured to detect the direction of the movement of the cable through the slot. A sensor assembly with a single sensor may requiring the initial status of the ultrasound probe to be manually configured by a user as being in the probe holder or out of the probe holder. Thereafter, the processor 104 tracks the current status of the ultrasound probe in the memory 106 by toggling the status of the ultrasound probe in the memory 106 as being placed in the probe holder or removed from the probe holder with each subsequent detection, by the sensor assembly, of the movement of the cable through the slot. The activation status of the ultrasound probe may be changed to an active state when the ultrasound probe is removed from the probe holder. The activation status of the ultrasound probe may be changed to an inactive state when placed in the probe holder. The processor 104 may be configured to update the memory 106 to reflect status changes, activation state changes, probe location changes, or changes in associations between probe ports and ultrasound probe.

As an example, when the first ultrasound probe 131 is connected to the first probe port 171 and the first ultrasound probe 131 is associated with the first probe holder 121, and the first ultrasound probe 131 is currently placed in the cradle (not shown in FIG. 1 or FIG. 2) of the first probe holder 121, and the processor 104 receives a signal from the sensor assembly 141 in response to detecting movement of the cable through the slot due to the removal of the ultrasound probe from the cradle of the first probe holder 121, the processor 104 is configured to track the removal of the first ultrasound probe by storing the status of the first ultrasound probe as being removed from the cradle in the memory 106. The activation state of the first ultrasound probe 131 may be set to an active state if each of the plurality of ultrasound probes (131, 132, 133, 134) is not currently in the active state. If the first ultrasound probe 131 is activated, the memory 106 is updated with the current configuration of the first ultrasound probe 131 as being out of the first probe holder 121 and being in the active state.

When the first ultrasound probe 131, is connected to the first probe port 171, and the first ultrasound probe 131 is associated with the first probe holder 121, and the first ultrasound probe 131 is set to the active state, and the first ultrasound probe 131 is placed into the cradle of the first probe holder 121, the processor 104 receives a signal from the sensor assembly 141 in response to detecting movement of the cable through the slot due to the first ultrasound probe 131 being placed in the cradle of the first probe holder 121. The processor 104 may set the activation state of the first ultrasound probe 131 to the inactive state. The memory 106 is updated with the current configuration of the first ultrasound probe 131 as being in the first probe holder 121 and being in the inactive state. The processor 104 may be configured to update the memory 106 to reflect status changes, activation state changes, probe location changes, or changes in associations between probe ports and ultrasound probe.

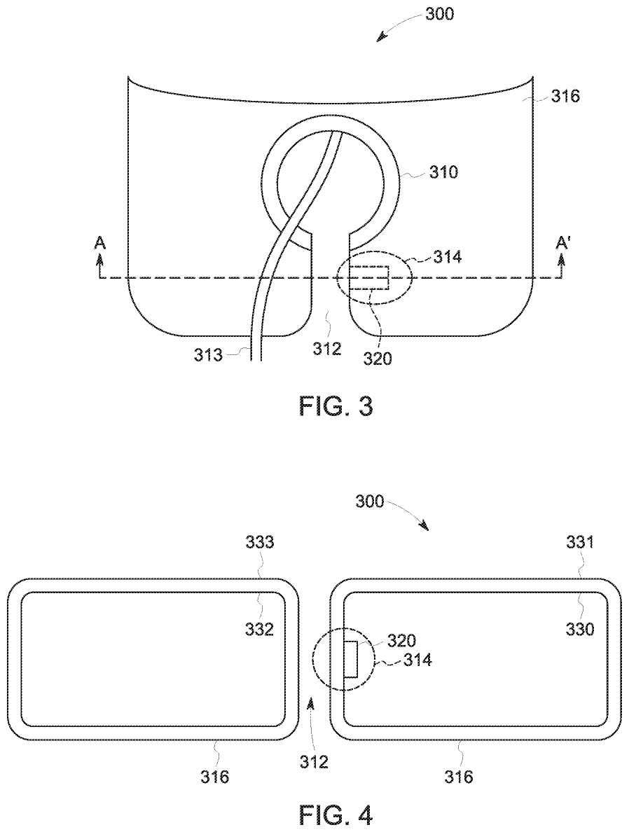

FIG. 3 is an illustration of a probe holder 300 in accordance with an exemplary embodiment. One or more of the plurality of probe holders (121, 122, 123, 124) shown in FIG. 2 may be configured in accordance with the embodiment shown in FIG. 3. The probe holder 300 includes a housing 316 that is shaped to define a cradle 310 for supporting an ultrasound probe, such as the first ultrasound probe 131. The housing 316 is further shaped to define a slot 312 to allow passage of the cable 313 through the slot 312 as the first ultrasound probe 131, is removed from or placed in the cradle 310 of the probe holder 300. A sensor assembly 314 is mounted to an interior surface of the housing 316 of the probe holder 300 in an area that defines the slot 312. The sensor assembly 314 may be secured to the housing 316 using adhesives, glue, welding, fusion solvents, or any other method capable of securing the sensor assembly 314 to the housing 316. In other embodiments, the sensor assembly 314 may be integrated into the housing 316 defining the slot 312, located on an exterior surface of the housing 316 within the slot 312, or located anywhere on the housing 316 of the probe holder 300 that enables the sensor assembly 314 to detect the movement of the cable 313 as the cable 313 passes through the slot 312. The sensor assembly 314 is in electronic communication with the processor 104. The sensor assembly 314 sends signals to the processor 104 based on the detection of the movement of the cable 313 through the slot 312 as the first ultrasound probe 131 is removed from or placed in the cradle 310.

The sensor assembly 314 includes a single sensor 320 The sensor 320 may be a capacitive sensor, an electromagnetic sensor, an inductive proximity sensor, an optical sensor, or any type of sensor capable of detecting the movement of the cable 313 through the slot 312 as the first ultrasound probe 131 is removed from or returned to the cradle 310. A signal is sent to the processor 104 from the sensor 320 with each detection of the cable 313 moving through the slot 312. According to embodiments where the sensor 320 is an optical sensor, the optical sensor may include both a transmitter and a received located on the same side of the slide 312. For example, the transmitter may transmit a signal, such as a beam of light. The beam of light may be in the optical range of wavelengths, in a wavelength shorter than the optical range, or in a wavelength longer than the optical range of wavelengths. The receiver is configured to receive the reflected beam of light in order to determine a distance. When the cable 313 is passed through the slot 312, the distance is shorter than where there is not a cable obscuring the slot. The processor 104 may use this information to detect the presence of the cable 313 even when both the transmitter and the receiver of an optical sensor are both located on the same side of the slot 312. The processor 104 may not be configured to detect the direction of the movement of the cable, requiring the initial status of the first ultrasound probe 131 to be manually configured by a user as being in the probe holder 300 or out of the probe holder 300. Thereafter, the processor 104 may be configured to track the current status of the first ultrasound probe 131 in the memory 106 by toggling the status of the first ultrasound probe 131 as being placed in the probe holder 300 or removed from the probe holder 300 with each subsequent detection of the movement of the cable 313 through the slot 312 by the sensor 320. The activation status of the first ultrasound probe 131 may be changed to an active state when the first ultrasound probe 131 is removed from the first probe holder 121, and the activation status may be changed to an inactive state when placed in the first probe holder 121. The processor 104 tracks status changes in the memory 106, including activation state changes, the position of an ultrasound probe with respect to a probe holder, and associating an ultrasound probe with a different probe port.

FIG. 4 illustrates a sectional view of the probe holder 300 as viewed along the dashed line A-A′ of FIG. 3. The sensor assembly 314 is mounted to an back side 330 of the housing 316 shaped to define a slot 312 of the probe holder 300. The sensor assembly 314 may be secured to the housing 316 using adhesives, glue, welding, fusion solvents, or any other method capable of securing the sensor assembly 314 to the housing 316.

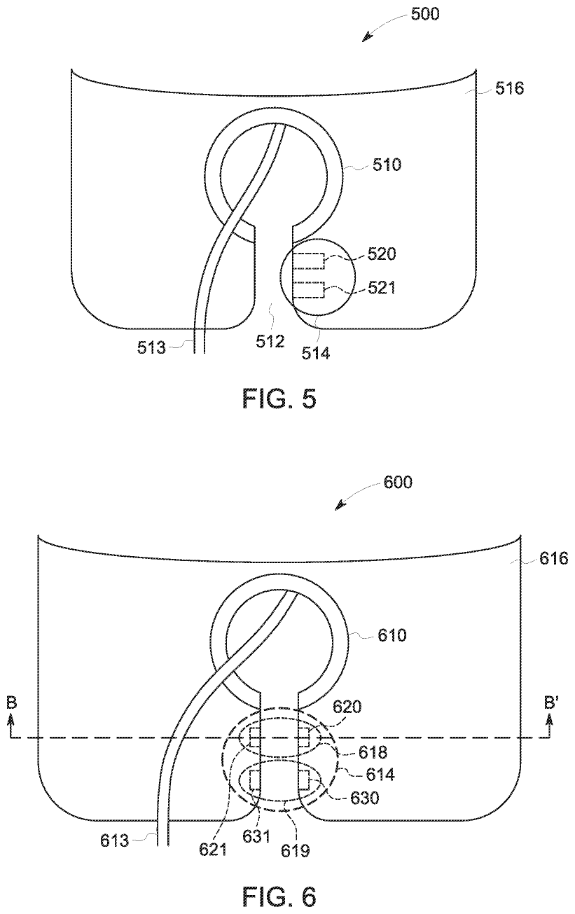

FIG. 5 is an illustration of a probe holder 500 in accordance with an exemplary embodiment. One or more of the plurality of probe holders (121, 122, 123, 124) shown in FIG. 2 may be configured in accordance with the embodiment shown in FIG. 5. The probe holder 500 includes a housing 516 that is shaped to define a cradle 510 for an ultrasound probe to rest upon, such as the first ultrasound probe 131. The housing 516 is further shaped to define a slot 512 to allow passage of the cable 513 through the slot 512 as a probe, such as the first ultrasound probe 131, is removed from or placed in the cradle 510 of the probe holder 500. A sensor assembly 514 is mounted to an interior surface of the housing 516 of the probe holder 500 in an area that defines the slot 512. In other embodiments, the sensor assembly 514 may be integrated into the housing 516 defining the slot 512, located on an exterior surface of the housing 516 within the slot 512, or located anywhere on the housing 516 of the probe holder 500 that enables the sensor assembly 514 to detect the movement of the cable 513 as the cable 513 passes through the slot 512. The sensor assembly 514 is in electronic communication with the processor 104. The sensor assembly 514 sends signals to the processor 104 based on the detection of the movement of the cable 513 through the slot 512 as the first ultrasound probe 131 is removed from or placed in the cradle 510.

The sensor assembly 514 includes a first sensor 520 and a second sensor 521. The first sensor 520 and the second sensor 521 are positioned on the same side of the housing 516 with respect to the slot 512. In other embodiments, the first sensor 520 and the second sensor 521 may be positioned on opposite sides of the housing 516 with respect to the slot 512. The first sensor 520 is positioned closer to the cradle 510 than the second sensor 521. The processor 104 may be configured to determine a direction of the movement of the cable 513 through the slot 512 based on an order of signals received from the first sensor 520 and the second sensor 521. The direction of the movement is into the cradle 510 when the signal is received from the second sensor 521 before the signal is received from the first sensor 520. The direction of the movement is outward from the cradle 510 when the signal is received from the first sensor 520 before the signal is received from the second sensor 521. The processor 104 is configured to control the activation state of the first ultrasound probe 131. If no other ultrasound probes (132, 133, 134) are in the active state, the activation state of the first ultrasound probe 131 may be set to the active state when the direction of the movement of the cable is outward from the cradle 510. The activation state of the first ultrasound probe 131 may be set to the inactive state when the direction of the movement of the cable 513 is inward to from the cradle 510. The processor 104 updates the memory 106 as status changes occur, including activation state changes, changes to the location of the first ultrasound probe 131 as in or out of the first probe holder 121, the first ultrasound probe 131 being added or removed from the probe port 171, and the first ultrasound probe 131 being returned to a different probe holder (122, 123, 124) from which it was removed.

According to an embodiment, the sensors 520 and 521 may be capacitive sensors and are configured to detect the direction of the movement of the cable 513 through the slot 512. In other embodiments, more than two sensors may be included. In other embodiments, the sensors 520 and 521 may be electromagnetic sensors, inductive proximity sensors, optical sensors, or any other type of sensor capable of being mounted to the housing 516 and detecting the movement of the cable 513 as the cable 513 passes through the slot 512.

FIG. 6 illustrates a probe holder 600 in accordance with an exemplary embodiment. One or more of the plurality of probe holders (121, 122, 123, 124) shown in FIG. 2 may be configured in accordance with the embodiment shown in FIG. 6. The probe holder 600 includes a housing 616 that is shaped to define a cradle 610 for an ultrasound probe, such as the first ultrasound probe 131. The housing 616 is further shaped to define a slot 612 to allow passage of the cable 613 through the slot 612 as the first ultrasound probe 131, for example, is removed from or placed in the cradle 610 of the probe holder 600. A sensor assembly 614 is positioned within openings (not shown in FIG. 6) of the housing 616 located within the portion of the housing 616 shaped to define the slot 612 of the probe holder 600. The sensor assembly 614 may be press-fit into the openings or mounted to the edges of the openings using adhesives, glue, welding, fusion solvents, or any other methods capable of securing the sensor assembly 614 to the edges of housing 616 defining the openings in the housing 616. The sensor assembly 614 is electronically connected to the processor 104. The sensor assembly 614 sends signals the processor 104 based on the detection of the movement of the cable 613 through the slot 612 as the first ultrasound probe 131 is removed from or returned to the cradle 610.

In accordance with the embodiment shown in FIG. 6, the sensor assembly 614 includes a first optical sensor 618 and a second optical sensor 619. The first optical sensor 618 includes a first optical transmitter 620 and a first optical receiver 621. The first optical transmitter 620 is located on one side of the housing 616. The first optical receiver 621 is located on the opposite side of the housing 616 with respect to the slot 612. The first optical transmitter 620 projects a first beam of light (not shown). The first beam of light is received by the first optical receiver 621. When there is a break in the first beam of light, the first optical receiver 621 sends a signal to the processor 104 indicating a detection of movement of the cable 613 through the slot 612. The second optical sensor 619 includes a second optical transmitter 630 and a second optical receiver 631. The second optical transmitter 630 is located on one side of the housing 616. The second optical receiver 631 is located on the opposite side of the housing 616 with respect to the slot 612. The second optical transmitter 630 projects a second beam of light (not shown). The second beam of light is received by the second optical receiver 631. When there is a break in the second beam of light, the second optical receiver 631 sends a signal to the processor 104 indicating a detection of movement of the cable 613 through the slot 612.

The processor 104 is configured to determine a direction of the movement of the cable based on an order of signals received from the first optical sensor 618 and the second optical sensor 619. The direction of the movement is inward towards the cradle 610 when the signal is received from the sensor second optical sensor 619 before the signal is received from the first optical sensor 618. The direction of the movement is outward from the cradle 610 when the signal is received from the first optical sensor 618 before the signal is received from the second optical sensor 619. When the direction of travel of the cable 613 is inward towards the cradle 610, the processor 104 may set the activation state of the first ultrasound probe 131 to the inactive state. When the direction of the movement of the cable is out from the cradle 610 the processor 104 may set the activation state of the first ultrasound probe 131 to the active state. As changes occur, the processor 104 updates the memory 106 to reflect the changes in status.

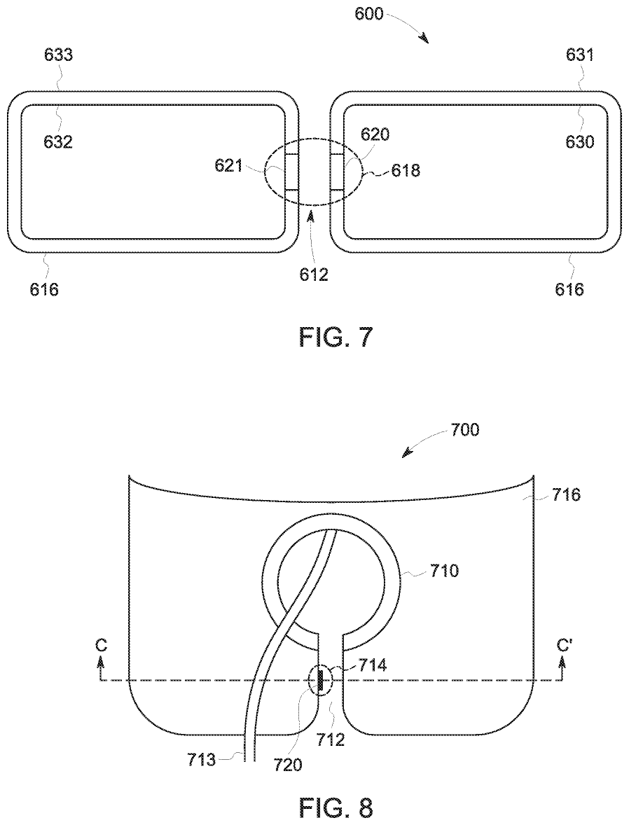

FIG. 7 illustrates a sectional view of the probe holder 600 as viewed along a dashed line B-B′ of FIG. 6. The first optical transmitter 620 and the first optical receiver 621 of the sensor 618 are mounted within the openings of the housing 616 (not shown) in the portion of the housing 616 shaped to define the slot 612 of the probe holder 600. The sensor 618 may be press-fit into the openings, mounted to the edges defined by the openings in the housing 616 using adhesives, glue, welding, fusion solvents, or any other methods capable of securing the sensor 618 to the housing 616.

FIG. 8 is an illustration of a probe holder 700 in accordance with an exemplary embodiment. One or more of the plurality of probe holders (121, 122, 123, 124) shown in FIG. 2 may be configured in accordance with the embodiment shown in FIG. 8. The probe holder 700 includes a housing 716 that is shaped to define a cradle 710 for an ultrasound probe to rest upon, such as the first ultrasound probe 131. The housing 716 is further shaped to define a slot 712 to allow passage of the cable 713 through the slot 712 as the first ultrasound probe 131, for example, is removed from or placed in the cradle 710 of the probe holder 700. A sensor assembly 714 is mounted to an exterior surface of the housing 716 of the probe holder 700 in an area that defines the slot 712. In other embodiments, the sensor assembly 714 may be integrated into the housing 716 defining the slot 712, located in an interior surface of the housing 716 within the slot 712, or located anywhere on the housing 716 of the probe holder 700 that enables the sensor assembly 714 to detect the movement of the cable 713 as the cable 713 passes through the slot 712. The sensor assembly 714 is electronically connected to the processor 104. The sensor assembly 714 sends signals to the processor 104 based on the detection of the movement of the cable 713 through the slot 712 as the first ultrasound probe 131 is removed from or placed in the cradle 710.

The sensor assembly 714 may be a single mechanical sensor 720. In accordance with other embodiments, the sensor 720 may be an electromagnetic sensor, an inductive proximity sensor, or any type of sensor capable of detecting the movement of the cable 713 through the slot 712 as the first ultrasound probe 131 is removed from or returned to the cradle 710. A signal is sent to the processor 104 from the sensor 720 with each detection of the cable 713 moving through the slot 712. The processor 104 may not be configured to detect the direction of the movement of the cable, requiring the initial status of the first ultrasound probe 131 to be manually configured by a user as being in the probe holder 700 or out of the probe holder 700. Thereafter, the processor 104 tracks the current status of the first ultrasound probe 131 in the memory 106 by toggling the status of the first ultrasound probe 131, in the memory 106, as being in the probe holder 700 or out of the probe holder 700 with each subsequent detection, by the sensor 720, of the movement of the cable 713 through the slot 712. The activation status of the first ultrasound probe 131 may be changed to an active state when the ultrasound probe 131 is removed from the first probe holder 121 and may be changed to an inactive state when placed in the first probe holder 121. The processor 104 updates the memory 106 as status changes occur, including activation state changes, the position of an ultrasound probe with respect to a probe holder, and associating an ultrasound probe with a different probe port.

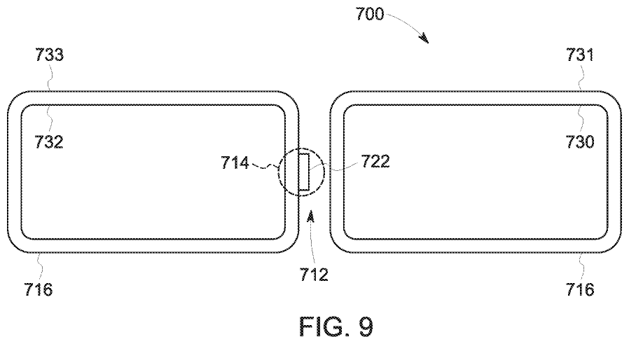

FIG. 9 is a sectional view of the probe holder 700 along a dashed line C-C′ shown in FIG. 8. A sensor assembly 714 is mounted to an exterior portion 733 of a housing 716 shaped to define a slot 712 of the probe holder 700. The sensor assembly 712 may be secured to the housing 716 using adhesives, glue, welding, fusion solvents, or any other method capable of securing the sensor assembly 714 to the housing 716.

FIGS. 3-9 show exemplary embodiments. It should be appreciated that other embodiments may use different configurations and types of sensors. Additionally, while many of the exemplary embodiments described hereinabove were described with respect to the first ultrasound probe 131, it should be appreciated by those skilled in the art that these embodiments may be configured to work with different probes, such as the second ultrasound probe 132, the third ultrasound probe 133, the fourth ultrasound probe 134, or any other ultrasound probe configured for use with the ultrasound imaging system 100.

Some embodiments may additionally use the input from the sensor assembly to control one or more scanning parameters for the ultrasound probe. For example, according to an embodiment, the processor 104 may store a set of scanning parameters for either each type of ultrasound probe or for each specific ultrasound probe. Scanning parameters may include parameters such as focal depth, pulse repetition frequency (PRF), line density, and frequency. According to one embodiment, the processor 104 may store the most-recently-used scanning parameter for each ultrasound probe used with the ultrasound imaging system. For example, after transitioning from an active state to an inactive state, as determined based on signals from the sensor assembly, the processor 104 may store the most-recently-used scanning parameters in a memory, such as the memory 106. Then, the next time that the activation state of that particular ultrasound probe changes from the inactive state to the active state, as determined based on signals from the sensor assembly, the processor 104 may access the memory 106 in order to retrieve the previously used scanning parameters for that particular ultrasound probe. As described previously, the processor 104 may store associations between the one or more ultrasound probes and one or more of the probe holders. The processor 104 may use these associations to identify the probe type of the probe being used (i.e. the probe for which the activation state transitioned from the inactive state to the active state) and to then use this information to access the previously used scanning parameters for that particular probe. According to other embodiments, the ultrasound imaging system may store a set of previously determined parameter settings each of a plurality of probe types in the memory 106.

According to an embodiment, the ultrasound imaging system may not have any parameters settings stored for a particular ultrasound probe. For situations where a new probe is connected to a probe port or the system is first powered on in the morning, it may be necessary for the user to manually input some or all of the values for the various parameters or default values will be used. According to an embodiment, the processor 104 may display a prompt on a display device requesting the input of one or more scanning parameters for an ultrasound probe and/or an identification of which ultrasound probe is being used. The identification of the probe being used may comprise a probe type, probe model or probe name, possibly selected from a displayed listing of probes recently used on the system.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. An ultrasound imaging system comprising:
an ultrasound probe including a cable;
a probe holder including a cradle for supporting the ultrasound probe and defining a slot for allowing passage of the cable;
a sensor assembly attached to the probe holder and configured to detect movement of the cable through the slot, wherein the sensor assembly includes at least one sensor; and
a processor configured to control an activation state of the ultrasound probe based on signals from the sensor assembly.

2. The ultrasound imaging system of claim 1, wherein the slot is elongated in a horizontal direction, and wherein it is necessary for the cable to pass through the slot when either removing the ultrasound probe from the cradle for scanning or returning the ultrasound probe to the cradle after scanning.

3. The system of claim 1, wherein the processor is configured to track whether the ultrasound probe is being removed from the cradle or placed into the cradle.

4. The ultrasound imaging system of claim 1, wherein the sensor is selected from an optical sensor, a capacitive sensor, an electromagnetic sensor, an inductive proximity sensor, and a mechanical sensor.

5. The ultrasound imaging system of claim 1, wherein the sensor comprises an optical sensor, and the optical sensor comprises a transmitter configured to project a beam of light and a receiver configured to receive the beam of light.

6. The ultrasound imaging system of claim 1, wherein the sensor assembly includes a first sensor and a second sensor, wherein the first sensor is positioned closer to the cradle than the second sensor, wherein the processor is configured to determine a cable direction based on an order of signals received from the first sensor and the second sensor, and wherein the processor is further configured to control the activation state of the ultrasound probe based on the cable direction.

7. The ultrasound imaging system of claim 1, further comprising a second probe holder comprising:
a second cradle for supporting the ultrasound probe and defining a second slot for allowing passage of the cable;
and a second sensor assembly attached to the second probe holder and configured to detect movement of the cable through the second slot.

8. The ultrasound imaging system of claim 7, wherein the processor is configured to store associations between the ultrasound probe and either the first probe holder or the second probe holder.

9. The ultrasound imaging system of claim 1, wherein the cradle of the probe holder is configured to support multiple different types of ultrasound probes.

10. The ultrasound imaging system of claim 1, wherein the cradle comprises a housing defining the slot, the housing including an exterior surface that defines the slot and an interior surface opposite of the exterior surface and wherein the sensor assembly is mounted to the housing.

11. The ultrasound imaging system of claim 10, wherein the sensor assembly is mounted on the interior surface of the housing.

12. The ultrasound imaging system of claim 10, wherein the sensor assembly is mounted on the exterior surface of the housing within the slot.

13. The ultrasound imaging system of claim 10, wherein the housing further defines an opening located within the slot, and wherein the sensor assembly is positioned within the opening.

14. The ultrasound imaging system of claim 1, wherein the ultrasound imaging system is configured to automatically set a plurality of scanning parameters for the ultrasound probe in response to having the activation state of the ultrasound probe change from an inactive state to an active state.

15. The ultrasound imaging system of claim 14, wherein the processor is configured to automatically set the plurality of scanning parameters based on a probe type of the ultrasound probe.

16. The ultrasound imaging system of claim 14, wherein the processor is configured to automatically set the plurality of scanning parameters to match previously used scanning parameters for the ultrasound probe.

17. The ultrasound imaging system of claim 1, wherein the processor is further configured to provide at least one of a prompt requesting input for one or more scanning parameters and an identification of the ultrasound probe being used.

18. A method for controlling an activation state of an ultrasound probe of an ultrasound imaging system, the ultrasound imaging system comprising a processor, a probe holder including a cradle for supporting the ultrasound probe and defining a slot for allowing passage of the cable, and a sensor assembly attached to the probe holder that is configured to detect movement of the cable through the slot, the method comprising:
receiving a signal from the sensor assembly in response to detecting movement of the cable through the slot due to either removing the ultrasound probe from the cradle or placing the ultrasound probe in the cradle; and
changing the activation state of the ultrasound probe based on the signal.

19. The method of claim 18, wherein said changing the activation state of the ultrasound probe comprises changing the activation state from an inactive state to an active state.

20. The method of claim 18, wherein said changing the activation state of the ultrasound probe comprises changing the activation state from an active state to an inactive state.

21. The method of claim 18, wherein the sensor assembly includes a first sensor and a second sensor, wherein the first sensor is positioned closer to the cradle than the second sensor, wherein the processor is configured to determine a cable direction based on an order of signals received from the first sensor and the second sensor, and wherein the processor is further configured to control the activation state of the ultrasound probe based on the cable direction.

22. The method of claim 18, further comprising providing a prompt requesting a user input to associate the ultrasound probe with one of a plurality of ports on the ultrasound imaging system.

23. The method of claim 18, wherein the sensor assembly is not configured to detect a cable direction and the processor is configured to track an ultrasound probe position.

24. The method of claim 18, wherein the ultrasound imaging system comprises a second probe holder and a second ultrasound probe, and wherein the processor is configured to save an association between the ultrasound probe and one of the probe holder and the second probe holder and between the second ultrasound probe and the other of the probe holder and the second probe holder.
